(19) United States
(12) Patent Application Publication
(10) Pub. No.: US 2010/0130460 A1
(43) Pub. Date: May 27, 2010

(54) TOPICAL GLUCOCORTICOSTEROID FORMULATIONS

(75) Inventors: Brian Albano, Huntington Station, NY (US); Robert J. Anderson, Stoney Brook, NY (US); Joseph A. Check, III, West Bayshore, NY (US); Weldon Crow, Freeport, NY (US)

Correspondence Address: SCHWEGMAN, LUNDBERG & WOESSNER, P.A., P.O. BOX 2938, MINNEAPOLIS, MN, 55402, US

(73) Assignee: Nycomed US Inc., Melville, NY (US)

(21) Appl. No.: 12/096,753

(22) PCT Filed: Dec. 8, 2006
(86) PCT No.: PCT/US2006/047027

§ 371 Date: Jan. 28, 2010

Related U.S. Application Data
(60) Provisional application No. 60/749,333, filed on Dec. 9, 2005.

(51) Int. Cl. A61K 31/569; A61P 17/04

(52) U.S. Cl. 514/180; 514/179

(57) ABSTRACT

Disclosed are topical formulations comprising an androstane steroid, propylene glycol, and benzyl alcohol in an amount effective to dissolve the androstane steroid compound, wherein the amount of benzyl alcohol is not effective to act as a penetration enhancer. The formulations are useful for the treatment of inflammatory and/or pruritic manifestations of corticosteroid-responsive dermatoses.

### RELATED APPLICATIONS

This application claims priority to U.S. Provisional Patent Application No. 60/749,333, filed Dec. 9, 2005, which is incorporated herein by reference.

### FIELD OF THE INVENTION

The present invention provides an androstane steroid topical dosage form, such as a cream, lotion, gel, ointment, shampoo, solution or transdermal patch comprising (a) propylene glycol and (b) benzyl alcohol in an amount effective to dissolve the androstane steroid.

### BACKGROUND OF THE INVENTION

Glucocorticosteroids, which have anti-inflammatory and anti-allergy properties, are well known and are widely used to treat conditions requiring an anti-inflammatory and/or anti-allergic response. One such class of glucocorticosteroids having such properties is androstane steroids of the type disclosed in U.S. Pat. No. 4,335,121, particularly fluticasone esters, and more particularly fluticasone propionate, namely 6α,9α-difluoro-17α(1-oxopropoxy)-11β-hydroxy-16α-methyl-3-oxo-androsta-1,4-diene-17β-carbothioic acid S-fluoromethyl ester, and derivatives thereof.

Fluticasone propionate cream and ointment are Class IV corticosteroid formulations currently marketed under the trademark CUTIVATE® for the relief of the inflammatory and pruritic manifestations of corticosteroid-responsive dermatoses. CUTIVATE® cream contains 0.05% fluticasone propionate for topical dermatologic use. Each gram of CUTIVATE® cream contains 0.5 mg of fluticasone propionate in a base of liquid paraffin, cetostearyl alcohol, isopropyl myristate, cetomacrogol 1000, propylene glycol, imidurea, sodium phosphate, citric acid monohydrate, and purified water. Propylene glycol is present in approximately 5% by weight.

CUTIVATE® ointment contains 0.005% fluticasone propionate. Each gram of CUTIVATE® ointment contains 0.05 mg fluticasone propionate in a base of propylene glycol, sorbitan sesquioleate, microcrystalline wax, and liquid paraffin. Propylene glycol is present in approximately 5% by weight. These formulations notwithstanding, there is a continuing need for additional corticosteroid formulations with higher potencies, as well as other advantageous properties.

It is desirable to increase the effectiveness of the active ingredient in such formulations. By increasing the vasoconstrictor activity, the effectiveness of the active ingredient is increased. International Patent Publication No. WO 00/24401 discloses that vasoconstrictor activity of fluticasone propionate lotion formulations is increased vis-à-vis fluticasone propionate cream formulations with decreased concentrations of occlusive agent, i.e., under 10% by weight. Nevertheless, addition of an occlusive agent, such as mineral oil or paraffin, is known to increase the vasoconstrictor potency of topical steroids. However, high concentrations of occlusive agents can cause the formulation to be unstable, and invert an oil-in-water emulsion to a water-in-oil emulsion that feels greasy.

In order to obtain high vasoconstrictor activity of such a formulation, while avoiding undue instability, and still have a relatively high level of occlusive agent in the product, International Patent Publication No. WO 02/13868 employs a specific type of surfactant system, namely one wherein the surfactant system has an HLB value ranging from about 7.0 to about 10.9 and wherein the surfactant system is present in the formulation in amounts ranging from about 0.25 to about 10.0% by weight.

United States Patent Application Publication Nos. 2003/0130247 A1, 2003/0176408 A1 and 2003/0186951 A1 disclose enhanced vasoconstriction activity of corticosteroids with combinations of at least two penetration enhancers, such that the ratio of penetration enhancers to the total of penetration enhancers, solvents, and emulsifiers is at least about 0.7:1.

There is still a continuing need for additional topical corticosteroid formulations with high vasoconstrictor activity.

### SUMMARY OF THE INVENTION

The present invention provides novel androstane steroid formulations, such as creams, lotions, gels, ointments, shampoo, solutions or transdermal patches, wherein the formulations include (a) propylene glycol, and (b) benzyl alcohol in an amount effective to dissolve the androstane steroid, but not in an amount effective to act as a penetration enhancer.

The present invention also provides a method of treating inflammation by applying to the skin of a patient in need of treatment an amount of an androstane steroid formulation, such as a cream, lotion, gel, ointment, shampoo, solution or transdermal patch of the present invention effective to treat said inflammation.

The present invention also provides a method of treating pruritus by applying to the skin of a patient in need of treatment an amount of an androstane steroid formulation, such as a cream, lotion, gel, ointment, shampoo, solution or transdermal patch of the present invention effective to treat said pruritus.

The invention also provides a topical composition and methods for the treatment of inflammatory and pruritic manifestations of atopic dermatitis, for example, corticosteroid-responsive dermatoses.

The androstane steroid compound in the formulations of the present invention is a compound of the formula:

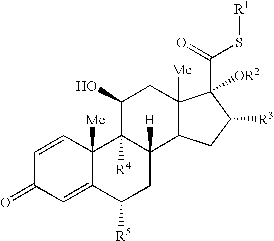

where R¹ is a fluoro-, chloro-, or bromo-methyl group, or a 2′-fluoroethyl group; R² is a group C(O)R⁶ where R⁶ is a C₁₋₃ alkyl group or OR² and R³ together form a 16α,17α-isopropylidenedioxy group; R³ is a hydrogen atom, a methyl group (which may be either the α- or β-configuration) or a methylene group; R⁴ is hydrogen, chlorine or fluorine atom; and R⁵ is a hydrogen or fluorine atom. The compounds of the invention include all stereoisomeric and diastereomeric derivative. In one embodiment, e.g., the formula above, the “solid wedge” symbol ▾ represents a single bond that is above the plane of the paper, and the “dashed line” symbol [symbol] represents a single bond that is behind the plane of the paper. Other steroids within the scope of the invention are disclosed in U.S. Pat. No. 4,335,121.

The present invention further provides a process for the preparation of formulations of the present invention which comprises dissolving the androstane steroid active ingredient in benzyl alcohol and adding the resultant solution to propylene glycol and optionally other components of the system which include, but are not limited to, surfactant(s), stiffening or thickening agent(s), wax(es), occlusive agent(s), emollient(s), preservative(s), base(s), water, buffer, or any combination thereof.

### BRIEF DESCRIPTION OF THE DRAWING

FIG. 1 is a graph of blanching response versus propylene glycol concentration for 0.05% fluticasone propionate creams using benzyl alcohol as a solvent. The 60% propylene glycol study was extrapolated from the data in the first study.

### DETAILED DESCRIPTION OF THE INVENTION

The present invention provides novel androstane steroid compositions, such as cream, lotion, gel, ointment, shampoo, solution, or transdermal patch formulations comprising (a) propylene glycol as the only penetration enhancer, and (b) benzyl alcohol in an amount effective to act as a solvent for and to dissolve the androstane steroid compound in vitro and in the steroid composition. The amount of benzyl alcohol required for use as a solvent is substantially less than the amount required when benzyl alcohol is used as a penetration enhancer. Androstane steroid creams, lotions, gels, ointments, shampoos, or transdermal patches in which the concentrations of propylene glycol are greater than about 40%, or greater than about 50%, by weight and in which benzyl alcohol is present in an amount effective to act as a solvent for and to dissolve the androstane steroid compound exhibit significantly higher vasoconstrictor activity than the corresponding formulations that contain concentrations of propylene glycol less than about 40%, or about 50%, by weight and higher concentrations of benzyl alcohol, such as when benzyl alcohol is used as a penetration enhancer. The vasoconstrictor activity can be so much higher that many of the androstane steroid creams, lotions, gels, ointments, shampoos, or transdermal patches of the present invention are considered Class I corticosteroids.

### DEFINITIONS

The following definitions are used, unless otherwise described:

The term “about” is intended to encompass variations in amounts of ingredients owing to variations in weighing and other measurement techniques, purity of ingredients, and the like, as would be known to the art worker. Such variations are often no more than about ±0.5%. The term “about” can indicate a variation of ±5 percent, or ±10 percent of the value specified; for example about 50 percent carries a variation from 45 to 55 percent; or the term can indicate ±1, 2, or 3 integers from the value specified.

The term “dissolve” as used with respect to benzyl alcohol, means that the steroid compound forms a clear solution in vitro with benzyl alcohol solvent. No solid material is out of solution when the clear solution is formed or comes out of solution when the benzyl alcohol solution is added to additional ingredients of the composition.

The term “effective amount” means a dosage sufficient to enhance the efficacy of treatment for the disease state or condition being treated. This will vary depending on the patient, the disease, and the treatment being effected.

The term “patient” refers to any animal, including mammals, preferably mice, rats, other rodents, rabbits, dogs, cats, swine, cattle, sheep, horses, or primates, and most preferably humans. The term may specify male or female or both, or exclude male or female.

The term “penetration enhancer” refers to a compound other than the active agent that is effective to increase the flux of the active agent through the stratum corneum.

The terms “treatment”, “treating”, and “treat” refer to administration of a compound for purposes including relieving the disease or condition, that is, causing the regression of clinical symptoms. The terms include preventing a pathologic condition from occurring (e.g. prophylaxis); inhibiting the pathologic condition or arresting its development; relieving a subject of the pathologic condition; and/or diminishing symptoms associated with the pathologic condition. The terms include reversing, preventing, ameliorating, or inhibiting an injury, disease-related condition, or a symptom of an injury or disease-related condition.

Corticosteroids are classified into seven categories according to their potencies; Class I includes the most potent, i.e., those with the highest bioavailability. The seven categories are:

Class I—Superpotent

Class II—Potent

Class III—Upper mid-strength

Class IV—Mid-strength

Class V—Lower mid-strength

Class VI—Mild

Class VII—Least potent.

Surprisingly, Applicants have found that when benzyl alcohol is used in an amount that will not cause penetration enhancement of the active agent, but is sufficient to dissolve the androstane steroid active ingredient, the potency of the resultant androstane steroid formulation increases markedly relative to formulations of the same active ingredient that do not employ benzyl alcohol as a solvent. The benzyl alcohol increases the solubility of the active ingredient in the vehicle, which results in a greater therapeutic effect at a substantially lower concentration compared to known formulations containing the same active ingredient.

Potency is determined via a vasoconstrictor assay (VCA), also called the skin blanching test, or the Stoughton-McKenzie test. The assay is based on the property of corticosteroids to produce blanching owing to vasoconstriction in the micro vasculature of the skin. This property is related to the amount of drug entering the skin, and thus it becomes a basis for assessing bioavailability and bioequivalence.

Although there are many forms of the VCA, the general method is based on topical application of a corticosteroid-containing formulation for a period of 6 to 16 hours in healthy human subjects, followed by visual estimation by a trained, blinded observer (or by use of a chromameter) of the degree of blanching, at a single time point, usually two hours, after removal of the formulation. Usually two in vivo studies are conducted—a pilot dose duration-response study and a pivotal bioequivalence study comparing test and reference products.

The pilot study was conducted solely with the reference listed drug. The comparison of test and reference products in the pivotal study is conducted at a dose duration approximately equal to the population ED₅₀ determined in the pilot study. The Food and Drug Administration, Center for Drug Evaluation and Research, has published Guidance for Industry, Jun. 2, 1995 (the contents of which are hereby incorporated by reference), setting forth guidance for the determination of in vivo bioequivalence.

The cream, lotion, gel, ointment, shampoo, solution or transdermal patch formulations of the present invention are useful for the relief of the inflammatory and pruritic manifestations of corticosteroid-responsive dermatoses such as atopic dermatitis, eczema, including atopic, infantile, and disco eczemas, purigo nodularis, neurodermatoses, including lichen simplex, lichen planus, seborrhoeic dermatitis, contact sensitivity reactions, discoid pupus erthematosus, insect bite reactions, prickly heat, erythema, population, scaling, erosion, oozing, crusting, bacterial infection, epidermolysis bullosa, psoriasis, erythema, hidradentis, suppurative warts, diaper rash, jock itch, or combinations of these conditions. The formulations can be applied topically to a patient in need of treatment for such condition(s).

Treatment with the creams, lotions, gels, ointments, shampoos, solutions or transdermal patches of this invention is usually accomplished by applying the creams, lotions, gels, ointments, shampoo, or transdermal patches to cover the affected area completely. The usual frequency of application is two to three times daily, although adequate maintenance therapy for some patients may be with less frequent application.

The topical formulations of the present invention typically comprise from about 0.0001 to about 1.0% by weight of active ingredient, preferably from about 0.01 to about 0.5%, e.g., from about 0.04 to about 0.2% by weight, for cream or lotion and gel formulations, and from about 0.0001 to about 1.0% by weight, preferably from about 0.005 to 0.2% by weight for ointment formulations. In certain specific embodiments, the composition includes about 0.03%, about 0.04%, about 0.05%, about 0.06%, about 0.07%, about 0.08%, about 0.09%, or about 1.0% of the active ingredient, or an amount in a range between any two of the aforementioned values.

The cream, lotion, and gel formulations are oil-in-water emulsions, and the ointments are non-aqueous dispersions in a base, wherein the active agent is dissolved. Suitable androstane steroid compounds useful as the active ingredient in the formulations of this invention are compounds of the formula:

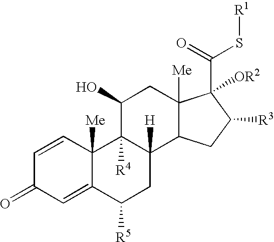

where R¹ is a fluoro-, chloro-, or bromo-methyl group or a 2′-fluoroethyl group; R² is a group COR⁶ where R⁶ is a C₁₋₃ alkyl group or OR² and R³ together form a 16α,17α-isopropylidenedioxy group; R³ is a hydrogen atom, a methyl group (which may be either the α- or β-configuration) or a methylene group; R⁴ is hydrogen, chlorine or fluorine atom; and R⁵ is a hydrogen or fluorine atom. The symbol ▾ represents a single bond that is above the plane of the paper, and the symbol [symbol] represents a single bond that is behind the plane of the paper.

Exemplary steroids include hydrocortisone and esters thereof, such as the butyrate, valerate, or probutate esters; betamethasone and esters thereof, such as the valerate ester; alclometasone dipropionate; halobetasol and esters thereof; desoximetasone; desonide; and the like. One specific active ingredient is fluticasone propionate, or a pharmaceutically acceptable salt or ester thereof.

Benzyl alcohol can be present in an amount sufficient to dissolve the androstane steroid compound, but not in an amount sufficient to act as a significant penetration enhancer (e.g., about 1% to about 3% by weight, or about 2% by weight). Moderate heat below the boiling point of benzyl alcohol may be applied, and stirring may be used to form a solution of the androstane steroid compound in benzyl alcohol.

For example, benzyl alcohol can be present in an amount of about 0.1% to about 20%, or from 0.1% to about 10%, by weight, of the total formulation. In one embodiment, benzyl alcohol can be present in about 0.2% to about 8%, by weight. In another embodiment, benzyl alcohol can be present in about 0.5% to about 6%. In yet another embodiment, benzyl alcohol can be present in about 0.5% to about 5%. In a further embodiment, benzyl alcohol can be present in about 1% to about 4%. In other embodiments, benzyl alcohol can be present in about 1.5% to about 3%. In yet other embodiments, benzyl alcohol can be present in about 2% of the composition. In various embodiments, these amounts are sufficient to dissolve the androstane steroid compound, but are not amounts sufficient to act as a penetration enhancer.

The composition percents recited herein refer to weight percent, unless otherwise stated. Other amounts of benzyl alcohol that can be employed include about 0.1%, about 0.5%, about 1%, about 1.5%, about 2%, about 2.5%, about 3%, about 4%, about 5%, about 7.5%, or about 10%, or any range in between any two of the aforementioned values.

Propylene glycol can be present generally in an amount of about 40% to about 90%, or about 50% to about 90%, by weight, of the total formulation. In other embodiments propylene glycol can be present at above about 50%, e.g., about 50.5% to about 75%, including about 55% to about 65%.

In one embodiment, propylene glycol can be present in about 40% to about 90%, by weight. In another embodiment, propylene glycol can be present in about 50% to about 90%. In yet another embodiment, propylene glycol can be present in about 50% to about 75%. In a further embodiment, propylene glycol can be present in about 55% to about 65%. In other embodiments, propylene glycol is present in about 50% by weight of the composition. In yet other embodiments, propylene glycol is present in about 60% by weight of the composition.

In various other embodiments, propylene glycol is present in about 45%, about 47.5%, about 48%, about 49%, about 49.5%, about 50%, about 50.5%, about 51%, about 52%, about 54%, about 55%, about 57.5%, about 58%, about 59%, about 60%, about 62%, about 63%, about 64%, about 65%, about 66%, or about 70%, by weight, of the composition, or any amount in between any two of the aforementioned percentages. In one specific embodiment, propylene glycol is present in about 50% of the composition, by weight, and in another specific embodiment, propylene glycol is present in about 60% of the composition, by weight.

The creams, lotions, gels, ointments, shampoos, solutions and transdermal patches may be formulated with conventional ingredients by methods known in the art. The formulations of the present invention are prepared by dissolving the androstane steroid active ingredient in benzyl alcohol and then adding the resulting solution to propylene glycol and other components of the system that may include, but are not limited to, surfactant(s), stiffening or thickening agent(s), wax(es), occlusive agent(s), emollient(s), preservative(s), base(s), and water or buffer, and the like. Transdermal patches may be made by methods known to the art.

Any suitable compatible surfactant(s) may be employed in the topical formulations of this invention. Examples of such surfactants include, but are not limited to, ceteareth-20 available as CETOMACROGOL® 1000, glycerol monostearate, glycerol distearate, glyceryl stearate, polyoxyethylene stearate, a blend of glyceryl stearate and PEG-100 stearate, (as ARLACEL 165), polysorbate 40, polysorbate 60, polysorbate 80, CETETH-20®, sorbitan monopalmitate, sorbitan monostearate, sorbitan monooleate, sorbitan sesquioleate, and mixtures thereof.

The amount of surfactant(s) employed in the formulations of the present invention is generally from about 0.5% to about 10% by weight. For the ointment formulations of the present invention, the amount of surfactant(s) is generally from about 0.5% to about 5.0% by weight, and for the cream formulations of the present invention, the amount of surfactant(s) is generally from about 1.0% to about 10% by weight.

Any suitable occlusive agent may be employed in the topical formulations of the present invention. Suitable occlusive agents include, but are not limited to, petrolatum, microcrystalline wax, beeswax, mineral oil, squalene, liquid paraffin, shea butter, carnauba wax, SEPIGEL® (a blend of isoparaffin/polyacrylamide/laureth-7), or mixtures thereof. The occlusive agent is preferably a wax and is present in the formulations of the present invention in an amount of from about 5.0% to about 30% by weight. For the ointment formulations of the present invention, the occlusive agent or wax component will generally be present in an amount of from about 20% to about 30% by weight, and for the cream or lotion formulations of this invention in an amount of from about 5.0% to about 20% by weight.

Any suitable emollient or skin conditioning agent may optionally be included in the topical formulations of the present invention. Suitable emollients include, but are not limited to, cholesterol, glycerine, glyceryl monostearate, isopropyl myristate, isopropyl palmitate, cetyl alcohol, cetostearyl alcohol, lanolin alcohols, or mixtures thereof. Optionally, dimethicone, mineral oil, or white soft paraffin may also be incorporated into the formulations in relatively small amounts to act as a skin conditioner.

The emollient or skin conditioning agent may be absent, or present in the topical formulations of the present invention in an amount of from about 0.01 to about 40% by weight. In the ointment formulations of the present invention, an emollient or skin conditioning agent may generally be absent, or present in an amount of from about 0.01 to about 10% by weight, and in the cream or lotion formulations of the present invention may generally be present in an amount of from about 2.0% to about 40.0% by weight.

The formulations of the present invention may also optionally include a buffer or neutralizing agent. Examples of suitable buffers include, but are not limited to, citric acid, lactic acid, oleic acid, sodium phosphate, water, triethanolamine, sodium citrate, hydrochloric acid, and the like. The buffering agent may be present in the composition in any suitable buffering effective amount. The gel formulations generally contain a base, such as for example, sodium hydroxide, triethanolamine, and the like. The gel formulations of the present invention also generally include a volatile solvent, such as, for example, ethanol, isopropanol and the like.

The formulations of the present invention preferably do not include antimicrobial (preservative) components, due to the preservative effects of propylene glycol. Antioxidants can be included, such as butylated hydroxyanisole, butylated hydroxytoluene, disodium edetate, citric acid, and the like. The antioxidant can be absent, or present in the formulations of the present invention in an amount from about 0.01 to about 0.6% by weight, preferably in an amount of from about 0.3% to about 0.6% by weight.

The formulations of the present invention optionally contain one or more thickening or stiffening agents. Suitable thickening or stiffening agents include, but are not limited to dimethicone, soft paraffin, aluminum stearate, stearyl alcohol, polyethylene glycols, wool fat, beeswax, carboxypolymethylene and cellulose derivatives, such as ethyl cellulose or hydroxypropyl methyl cellulose, and/or glyceryl monostearate. The thickening or stiffening agents are absent, or present in the composition generally in an amount from about 0.0 to 10% by weight, preferably from about 0.1% to about 10% by weight, and more preferably in an amount of from about 0.1% to about 5% by weight.

The concentration of active ingredient in the cream formulations ranges from about 0.0001% to about 1.0% by weight. The cream formulations of the present invention are generally prepared by dissolving a steroid active ingredient, e.g., fluticasone propionate, in benzyl alcohol. The benzyl alcohol solution of steroid active ingredient is then added at ambient or elevated temperatures to the remaining ingredients of the formulation, comprising greater than 50% by weight propylene glycol, and allowed to cool if necessary.

The ointment formulations are generally prepared in the following manner: the steroid active ingredient, e.g., fluticasone propionate, is dissolved in benzyl alcohol and then added to propylene glycol. The total amount of propylene glycol in these ointment formulations is greater than about 50% by weight. This solution can then be dispersed in an oil base containing a surfactant and wax. The ointment base can comprise polyethylene glycol. The wax may be a single component or a combination of waxes with different physical properties. Optionally, the formulation may contain an emollient and a penetration enhancer.

Lotions and gels are prepared in a conventional manner. One or more thickening agents can be used to achieve the consistency of a lotion or gel. Cosmetic preference or stability considerations will dictate selection of the thickening agent(s).

While it is known that propylene glycol can increase vasoconstrictor activity of corticosteroid formulations not containing propylene glycol by acting as a penetration enhancer, there is no cause and effect relationship known between high concentrations of propylene glycol and Class I corticosteroid potency. Thus, for example, Synalar® topical solution contains 99.99% propylene glycol, but the solution is a Class IV formulation, and Lidex® cream contains 67.43% propylene glycol, but the cream is a Class II formulation.

In addition, high concentrations of propylene glycol as the sole penetration enhancer are known not to produce vasoconstrictor activities as high as propylene glycol in combination with other penetration enhancers, such as dimethyl isosorbide or diisopropyl adipate. Therefore, it is surprising and unexpected that a Class I formulation would result either by increasing the concentration of propylene glycol in a formulation of a lower potency class when the formulation already contains propylene glycol, or by adding high concentrations of propylene glycol as the sole penetration enhancer to formulations not containing any propylene glycol.

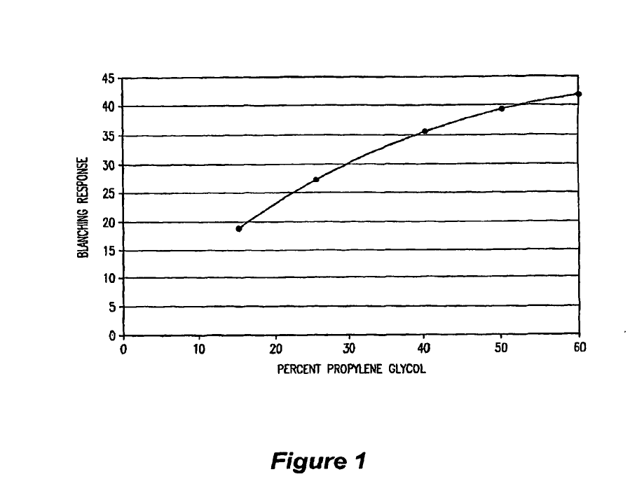

As shown in FIG. 1, the blanching response of 0.05% fluticasone propionate creams using benzyl alcohol as a solvent increases significantly with increased propylene glycol concentrations. The blanching response at a 60% propylene glycol concentration is more than twice the blanching response at a 15% propylene glycol concentration.

Heretofore, benzyl alcohol has been used in the art as a penetration enhancer and as a preservative/antioxidant. When used as a preservative/antioxidant, it is usually employed to the extent of about 1% by weight. Much larger concentrations of benzyl alcohol are required for benzyl alcohol to function as a penetration enhancer. For example, depending on the ingredients of the composition, benzyl alcohol present in greater than about 10%, or greater than about 20%, by weight, may be required to function as a penetration enhancer.

The invention will now be illustrated by the following non-limiting examples. Exemplary cream formulations of this invention include the following compositions. In these exemplary compositions, fluticasone propionate is used as the exemplary androstane steroid. However, it should be recognized that the compositions may contain any androstane steroid. It will be apparent to those skilled in the art that many modifications thereof may be practical without departing from the purpose and intent of this disclosure.

### EXAMPLES SECTION

### Example 1

### 0.04% Fluticasone Propionate Cream Formulation with Benzyl Alcohol and 50% Propylene Glycol

| Component | % (w/w) |
|---|---|
| Fluticasone propionate | 0.04 |
| Stearyl alcohol | 3.00 |
| Cetyl alcohol | 3.00 |
| Sorbitan monostearate | 0.30 |
| Mineral oil | 10.00 |
| Polysorbate 60 | 3.20 |
| Propylene glycol | 50.00 |
| Benzyl alcohol | 2.00 |
| Purified water | 28.46 |

### Example 2

### 0.05% Fluticasone Propionate Cream Formulation with 50% Propylene Glycol

| Component | % (w/w) |
|---|---|
| Fluticasone propionate | 0.05 |
| Stearyl alcohol | 3.00 |
| Cetyl alcohol | 3.00 |
| Sorbitan monostearate | 0.30 |
| Mineral oil | 10.00 |
| Polysorbate 60 | 3.20 |
| Propylene glycol | 50.00 |
| Benzyl alcohol | 2.00 |
| Purified water | 28.45 |

### Example 3

### 0.05% Fluticasone Propionate Cream Formulation with Benzyl Alcohol and 60% Propylene Glycol

| Component | % (w/w) |
|---|---|
| Fluticasone propionate | 0.05 |
| Stearyl alcohol | 3.00 |
| Cetyl alcohol | 3.00 |
| Sorbitan monostearate | 0.30 |
| Mineral oil | 10.00 |
| Polysorbate 60 | 3.20 |
| Propylene glycol | 60.00 |
| Benzyl alcohol | 2.00 |
| Purified water | 18.45 |

### Example 4

### 0.06% Fluticasone Propionate Cream Formulation with Benzyl Alcohol and 50% Propylene Glycol

| Component | % (w/w) |
|---|---|
| Fluticasone propionate | 0.06 |
| Stearyl alcohol | 3.00 |
| Cetyl alcohol | 3.00 |
| Sorbitan monostearate | 0.30 |
| Mineral oil | 10.00 |
| Polysorbate 60 | 3.20 |
| Propylene glycol | 50.00 |
| Benzyl alcohol | 2.00 |
| Purified water | 28.44 |

### Preparation Example 5

A topical 0.05% fluticasone propionate cream containing 60% propylene glycol was prepared as follows:

A 1-liter Pyrex® glass beaker was equipped with a laboratory mixer with a stainless steel propeller and shaft and placed on a hotplate. A second 600 mL support beaker was equipped similarly. A 100 mL beaker was equipped with a magnetic stirring bar and placed upon a magnetic stirrer.

To the 1-liter beaker were added 15.0 g of stearyl alcohol NF, 15.00 g of cetyl alcohol NF, 1.5 g of sorbitan monostearate NF, and 50.0 g of mineral oil USP. The mixture was heated to 60-65° C. with mixing at 150-350 rpm until melted and homogeneous. Heating and mixing was continued until used in the following step.

To the support beaker were added 92.25 g of purified water USP, 16.0 g of polysorbate 60 NF, and 300 g of propylene glycol. The mixture was heated to 60-65° C. with mixing at 150-350 rpm until clear and homogeneous. With the contents of both beakers at 60-65° C., the solution from the support beaker was added to the contents of the 1-liter beaker. During the transfer, counter-rotating mixing was continued at 150-350 rpm. The combined mixture was cooled to 40-45° C. while mixing was continued at 150-350 rpm.

To the 100 mL beaker were added 10.0 g benzyl alcohol NF and 0.25 g fluticasone propionate. The mixture was heated to 40-45° C. with mixing with a magnetic stirring bar and magnetic stirrer. Mixing was continued until the fluticasone propionate was dissolved. The contents were then added to the contents of the 1-liter beaker at 40-45° C. with mixing at 150-350 rpm. The mixture was then cooled to 25-30° C. with mixing at 150-350 rpm and transferred at 25-30° C. to suitable containers.

### Example 6

The fluticasone propionate creams prepared above were tested against Topicort® LP Emollient Cream (0.05%), a Class III corticosteroid formulation, and Ultravate® Cream (0.05%), a Class I corticosteroid formulation, in a vasoconstrictor assay, using standard protocols (Study 1). The two 0.05% fluticasone propionates creams were tested versus Ultravate® and Topicort® in a different subject population than the 0.04% and 0.06% concentrations (Study 2). Also included in both assays as a control was a vehicle for fluticasone propionate ointment.

The results were as follows:

| Mean Skin | |
|---|---|
| Bleaching Score | |
| (Chromameter) | Study 1 Medication |
| 0.417 | Fluticasone propionate ointment vehicle (placebo) |
| 1.528 | Topicort ® LP Emollient Cream (0.05%) (Class III) |
| 2.083 | Ultravate ® Cream (0.05%) (Class I) |
| 2.056 | Fluticasone propionate cream (0.05%), benzyl alcohol |
| | as solvent, with 50% propylene glycol |
| 2.194 | Fluticasone propionate cream (0.05%), benzyl alcohol |
| | as solvent, with 60% propylene glycol |

| Mean Skin | |
|---|---|
| Bleaching Score | |
| (Visual) | Study 2 Medication |
| 0.74 | Fluticasone propionate ointment vehicle (placebo) |
| 1.40 | Aclovate ® Cream (aclometasone dipropionate) |
| | (0.05%) |
| 1.57 | Topicort ® LP Emollient Cream (desoximetasone) |
| | (0.05%) (Class III) |
| 1.89 | Ultravate ® Cream (halobetasol propionate) (0.05%) |
| | (Class I) |
| 2.46 | Fluticasone propionate cream (0.04%), benzyl alcohol |
| | as solvent with 50% propylene glycol |

The visual scoring system (Study 2) remains the standard by which the currently available topical corticosteroids achieve their potency rankings. The primary efficacy variable was the skin blanching score measured on a four-point ordinal scale. The data were analyzed for mean differences among treatments using a randomized blocks analysis of variance or a nonparametric analog using the ranks of the scores with Subject as the blocking variable. Within this analysis, pairwise comparisons of the mean visual assessment scores were performed using the Ryan-Einot-Gabriel-Welsch Multiple Range Test (REGWQ) which controls the experiment wise Type I error rate at 5% under the complete null hypothesis. The null hypothesis states that the treatment blanching score means are equal to each other.

Fluticasone propionate cream (0.04%, 0.05% and 0.06%) prepared with benzyl alcohol as solvent and with 50% and 60% propylene glycol had vasoconstrictive properties that were consistent with those of a Class I corticosteroid (FIG. 1). They were more vasoconstrictive than Topicort® and not statistically different from Ultravate®, a Class I corticosteroid formulation. Commercially available 0.05% Cutivate® (Fluticasone propionate) cream and ointment are less potent Class III formulations. Therefore, the fluticasone propionate formulation of the present invention is markedly more potent relative to other known formulations of the same active ingredient.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the statements of the invention. It will be readily apparent to one skilled in the art that varying substitutions and modifications may be made to the invention disclosed herein without departing from the scope and spirit of the invention.

The invention described illustratively herein suitably may be practiced in the absence of any element or elements, or limitation or limitations, which is not specifically disclosed herein as essential. The methods and processes described illustratively herein suitably may be practiced in differing orders of steps, and that they are not necessarily restricted to the orders of steps indicated herein or in the statements of the invention.

As used herein and in the appended claims, the singular forms “a,” “an,” and “the” include plural reference unless the context clearly dictates otherwise. Thus, for example, a reference to “a topical formulation” includes a plurality of such formulations, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as set forth in the appended statements of the invention. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended statements of the invention.

The invention has been described broadly and generically herein. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein.

All patents and publications referenced or mentioned herein are indicative of the levels of skill of those skilled in the art to which the invention pertains, and each such cited patent or publication is hereby incorporated by reference to the same extent as if it had been incorporated by reference in its entirety individually or set forth herein in its entirety. Applicants reserve the right to incorporate physically into this specification any and all materials and information from any such cited patents or publications.

1. A topical composition for the treatment of inflammatory or pruritic manifestations of corticosteroid-responsive dermatoses comprising:
(a) an amount of an androstane steroid compound effective to treat said inflammatory or pruritic manifestations;
(b) propylene glycol in an amount greater than about 40% by weight of the total formulation; and
(c) benzyl alcohol in an amount effective to dissolve the androstane steroid compound;
wherein the amount of benzyl alcohol is not effective to act as a penetration enhancer.

2. A composition according to claim 1 wherein the androstane steroid is a compound of the formula: 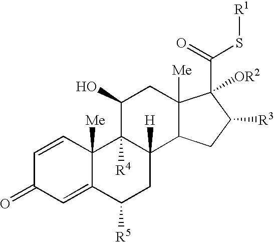
wherein
R¹ is a fluoro-, chloro-, or bromo-methyl group or a 2′-fluoroethyl group;
R² is a group COR⁶ where R⁶ is a C₁₋₃ alkyl group, or OR² and R³ together form a 16α,17α-isopropylidenedioxy group;
R³ is a hydrogen atom, a methyl group, or a methylene group;
R⁴ is a hydrogen, chlorine, or fluorine atom; and
R⁵ is a hydrogen or fluorine atom;
or a pharmaceutically acceptable salt thereof.

3. A composition according to claim 1 wherein the androstane steroid compound is fluticasone propionate.

4. A composition according to claim 1 wherein propylene glycol is present in an amount of about 50% to about 75% by weight of the total formulation.

5. A composition according to claim 1 wherein propylene glycol is present in an amount of about 55% to about 65% by weight of the total formulation.

6. A composition according to claim 1 wherein benzyl alcohol is present in an amount of about 0.1% to about 10% by weight of the total formulation.

7. A composition according to claim 1 wherein benzyl alcohol is present in an amount of about 0.5% to about 5% by weight of the total formulation.

8. A composition according to claim 1 wherein the composition is a cream.

9. A composition according to claim 1 wherein the composition is a solution or a lotion.

10. A composition according to claim 1 wherein the composition is a gel.

11. A composition according to claim 1 wherein the composition is an ointment.

12. A composition according to claim 1 wherein the composition is part of a transdermal patch.

13. A composition according to claim 1, wherein the composition has Class I corticosteroid potency.

14. A method of treating inflammation comprising applying to the skin of a patient in need of treatment an effective anti-inflammatory amount of a composition which comprises:
(a) an androstane steroid compound;
(b) propylene glycol in an amount greater than about 40% by weight of the total formulation; and
(c) benzyl alcohol in an amount effective to dissolve the androstane steroid compound;
wherein the amount of benzyl alcohol is not effective to act as a penetration enhancer.

15. The method of claim 14 wherein the androstane steroid is a compound of the formula: 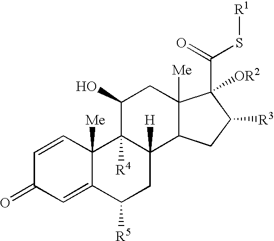
wherein
R¹ is a fluoro-, chloro-, or bromo-methyl group or a 2′-fluoroethyl group;
R² is a group COR⁶ where R⁶ is a C₁₋₃ alkyl group, or OR² and R³ together form a 16α,17α-isopropylidenedioxy group;
R³ is a hydrogen atom, a methyl group, or a methylene group;
R⁴ is hydrogen, chlorine or fluorine atom; and
R⁵ is a hydrogen or fluorine atom;
or a pharmaceutically acceptable salt thereof.

16. A method according to claim 14 wherein the androstane steroid compound is fluticasone propionate.

17. A method according to claim 14 wherein propylene glycol is present in an amount of about 51% to about 75% by weight of the total formulation.

18. A method according to claim 14 wherein propylene glycol is present in an amount of about 55% to about 65% by weight of the total formulation.

19. A method according to claim 14 wherein benzyl alcohol is present in an amount of about 0.1% to about 10% by weight of the total formulation.

20. A method according to claim 14 wherein benzyl alcohol is present in an amount of about 0.5% to about 5% by weight of the total formulation.

21. A method according to claim 14 wherein the composition is a cream.

22. A method according to claim 14 wherein the composition is solution or is a lotion.

23. A method according to claim 14 wherein the composition is a gel.

24. A method according to claim 14 wherein the composition is an ointment.

25. A method according to claim 14 wherein the composition is part of a transdermal patch.

26. A method of treating pruritus which comprises applying to the skin of a patient in need of treatment an effective anti-pruritic amount of a composition which comprises:
(a) an androstane steroid compound;
(b) propylene glycol in an amount greater than about 40% by weight of the total formulation; and
(c) benzyl alcohol in an amount effective to dissolve the androstane steroid compound;
wherein the amount of benzyl alcohol is not effective to act as a penetration enhancer.

27. The method of claim 26 wherein the androstane steroid is a compound of the formula: 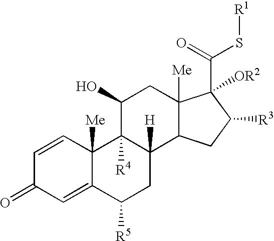
wherein
R¹ is a fluoro-, chloro-, or bromo-methyl group or a 2′-fluoroethyl group;
R² is a group COR⁶ where R⁶ is a C₁₋₃ alkyl group, or OR² and R³ together form a 16α,17α-isopropylidenedioxy group;
R³ is a hydrogen atom, a methyl group, or a methylene group;
R⁴ is hydrogen, chlorine or fluorine atom; and
R⁵ is a hydrogen or fluorine atom;
or a pharmaceutically acceptable salt thereof.

28. A method according to claim 26 wherein the androstane steroid compound is fluticasone propionate.

29. A method according to claim 26 wherein propylene glycol is present in an amount of about 40% to about 75% by weight of the total formulation.

30. A method according to claim 26 wherein propylene glycol is present in an amount of about 55% to about 65% by weight of the total formulation.

31. A method according to claim 26 wherein benzyl alcohol is present in an amount of about 0.1% to about 10% by weight of the total formulation.

32. A method according to claim 26 wherein benzyl alcohol is present in an amount of about 0.5% to about 5% by weight of the total formulation.

33. A method according to claim 26 wherein the composition is a cream.

34. A method according ton claim 26 wherein the composition is solution or is a lotion.

35. A method according to claim 26 wherein the composition is a gel.

36. A method according to claim 26 wherein the composition is an ointment.

37. A method according to claim 26 wherein the composition is part of a transdermal patch.

38. A composition according to claim 2 wherein R³ is in the α-configuration.

39. A composition according to claim 2 wherein R³ is in the β-configuration.

40. A method according to claim 15 or 27 wherein R³ is in the α-configuration.

41. A method according to claim 15 or 27 wherein R³ is in the β-configuration.

42-46. (canceled)
